(19) United States
(12) Patent Application Publication
(10) Pub. No.: US 2012/0302851 A1
(43) Pub. Date: Nov. 29, 2012

(54) PHYSIOLOGICAL SIGNAL MEASURING APPARATUS AND METHOD WITH IDENTIFICATION FUNCTION

(76) Inventors: Shu-Hung Lin, Hsichih (TW); Yao-Tsung Chang, Hsichih (TW); Chia-Hsien Li, Hsichih (TW); Pai-Yang Lin, Hsichih (TW); Shun-Chi Chung, Hsichih (TW)

(21) Appl. No.: 13/420,895

(22) Filed: Mar. 15, 2012

(30) Foreign Application Priority Data
May 27, 2011 (TW) 100118703

(51) Int. Cl. A61B 5/145; A61B 5/0402; A61B 5/021; A61B 5/00

(52) U.S. Cl. 600/365; 600/300; 600/509; 600/485

(57) ABSTRACT

A physiological signal measuring apparatus and method with identification function are provided. The physiological signal measuring apparatus with identification function includes a measuring module, an identifying module, and a transmitting module. The measuring module obtains a physiological signal and produces a physiological datum according to the physiological signal. The identifying module obtains an identification datum. The transmitting module is electrically connected to the measuring module and the identifying module for transmitting the physiological datum and the identification datum to a computer.

### BACKGROUND OF THE INVENTION

1. Field of the Invention

The present invention relates to a physiological signal measuring apparatus and method with identification function, and more particularly, to a physiological signal measuring apparatus and method with identification function in performing identification on a device proper or on an external electronic device.

2. Description of the Prior Art

In a modern society, due to medical advancements, people nowadays are attaching increasingly great importance to personal health and thus often need to measure physiological signals related to blood pressure, blood sugar, or body fat, etc. Due to technological advancements, measured physiological signals are not necessarily recorded on conventional paper or stored in a storage device of a computer, but can be directly stored in a cloud-based database to enable medical institutions to better perform real-time patient follow-up.

The elderly account for the majority of the numerous patients who need to be monitored in real time. The elderly are seldom good at operating a computer. Hence, increased ease of operating a computer will result in more convenient and less erroneous measurement of physiological signals. Measuring physiological signals and identifying a user identity concurrently can reduce the required manpower of medical care institutions, simplify the way in which the user operates a computer (especially computer operations performed by the elderly or users unfamiliar with the use of computers), and prevent a measurement record from being wrongly entered because of a mistake made by the user in the course of operation.

In addition to common means of recognition, such as fingerprint and retina recognition, identity recognition technology applies to identity recognition, using recognition means such as a palmprint, face, iris, DNA, or electrocardiography (ECG) waveforms. In this regard, many academic institutions, both local and overseas, study the identity recognition rate achievable by ECG waveforms, and in consequence, the ECG waveform-based identity recognition rate is confirmed to be above 90% for various algorithms.

Accordingly, the present invention provides a physiological signal measuring apparatus and method with identification function to measure a physiological signal and identify a user identity concurrently with a view to overcoming the drawbacks of the prior art.

### SUMMARY OF THE INVENTION

It is a primary objective of the present invention to provide a physiological signal measuring apparatus with identification function.

Another primary objective of the present invention is to provide a physiological signal measuring method with identification function.

In order to achieve the above and other objectives, the physiological signal measuring apparatus with identification function of the present invention comprises a measuring module, an identifying module, and a transmitting module. The measuring module obtains a physiological signal and produces a physiological datum according to the physiological signal. The identifying module obtains an identification datum. The transmitting module is electrically connected to the measuring module and the identifying module for transmitting the physiological datum and the identification datum to a computer.

In order to achieve the above and other objectives, the physiological signal measuring method with identification function of the present invention comprises the steps of: receiving a physiological datum and an identification datum; determining whether the identification datum is present in an identity identification database, so as to perform one of the two steps of: providing a user identity datum corresponding to the identification datum and storing the identification datum and the user identity datum in the identity identification database if it is determined that the identification datum is not present in the identity identification database; or obtaining the user identity datum corresponding to the identification datum if it is determined that the identification datum is present in the identity identification database; and storing the physiological datum in a corresponding physiological data region in a physiological database according to the user identity datum.

### BRIEF DESCRIPTION OF THE DRAWINGS

To render the above and other objectives, features, and advantages of the present invention more salient and comprehensible, the present invention are hereunder illustrated with specific embodiments in conjunction with the accompanying drawings, in which:

FIG. 1 is a schematic view of a physiological signal measuring apparatus with identification function according to the first embodiment of the present invention;

FIG. 2 is a schematic view of the physiological signal measuring apparatus with identification function according to the second embodiment of the present invention;

FIG. 3 is a flowchart of a physiological signal measuring method with identification function according to the present invention;

FIG. 4 is a schematic view of a physiological database and an identity identification database according to the present invention; and

FIG. 5 is a schematic view of an operating interface of the physiological signal measuring apparatus with identification function according to the present invention.

### DETAILED DESCRIPTION OF THE EMBODIMENTS

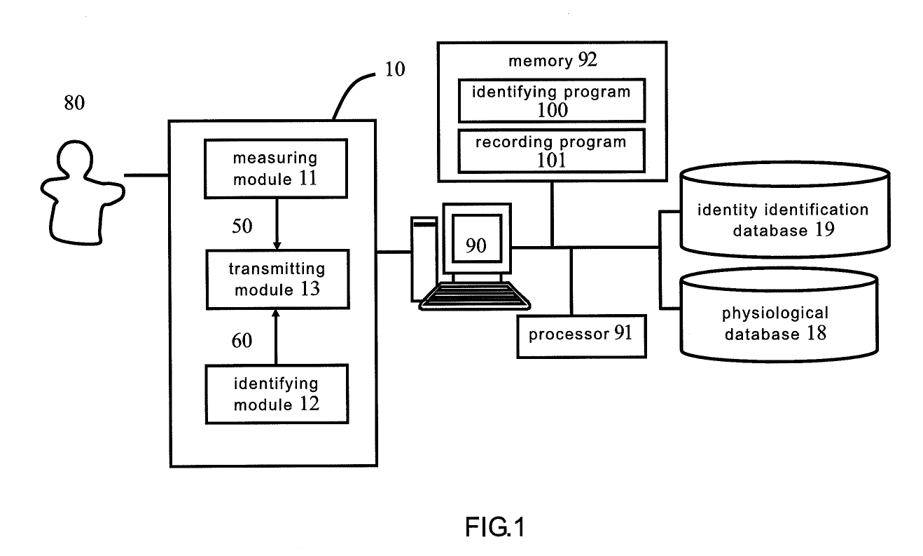

Referring to FIG. 1, there is shown a schematic view of a physiological signal measuring apparatus with identification function according to the first embodiment of the present invention. In this embodiment, a physiological signal measuring apparatus with identification function (10) can be connected to an external electronic device 90, such that the external electronic device 90 performs identity identification. The physiological signal measuring apparatus with identification function (10) comprises a measuring module 11, an identifying module 12, and a transmitting module 13. The external electronic device 90 comprises a processor 91 and a memory 92. An identifying program 100 and a recording program 101 are stored in the memory 92. The external electronic device 90 is connected to a physiological database 18 and an identity identification database 19. After obtaining a physiological signal (such as blood pressure, blood sugar, or heartbeat) of a user 80 through the measuring module 11, the physiological signal measuring apparatus with identification function (10) produces a physiological datum 50 (such as a blood pressure-related datum, a blood sugar-related datum, or an electrocardiogram-related datum) according to the physiological signal, obtains an identification datum 60 (such as a fingerprint-related datum or an electrocardiogram-related datum) of the user 80 through the identifying module 12, and transmits the physiological datum 50 and the identification datum 60 to the external electronic device 90 via the transmitting module 13. The processor 91 of the external electronic device 90 executes the identifying program 100 in the memory 92, searches the identity identification database 19 to identify the identity of the user 80 according to the identification datum 60, and then executes the recording program 101 to store the physiological datum 50 in a corresponding physiological data region in the physiological database 18. In this embodiment, the external electronic device 90 is a personal computer, though the disclosure of the present invention is not limited thereto, because the external electronic device 90 can also be a PDA or a smart phone.

A point to note is that the application of the physiological signal measuring apparatus with identification function (10) of the present invention is not limited to connection with the external electronic device 90. The user 80 can also directly use the physiological signal measuring apparatus with identification function (10) to measure the physiological signal and identify a user identity concurrently, without using the external electronic device 90.

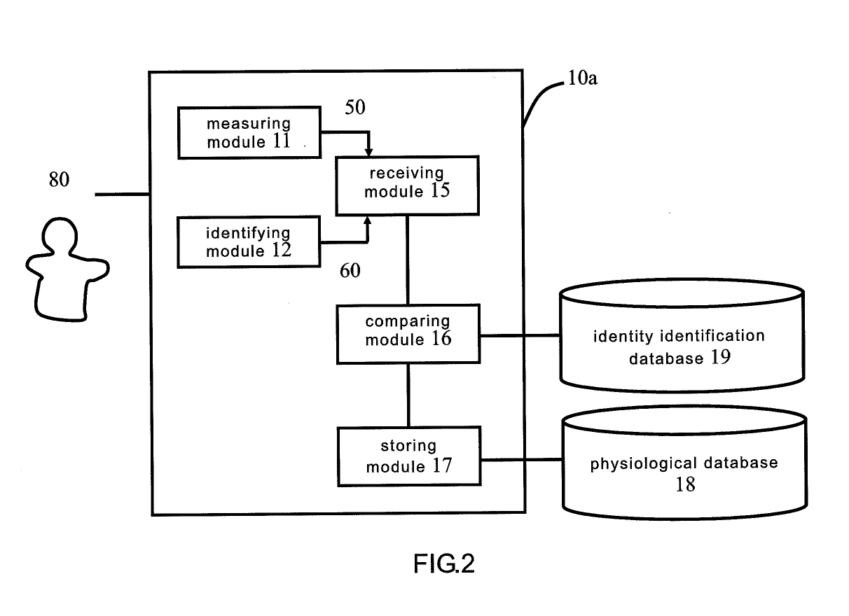

Referring to FIG. 2, there is shown a schematic view of a physiological signal measuring apparatus with identification function (10a) according to the second embodiment of the present invention. In this embodiment, the user 80 can directly use the physiological signal measuring apparatus with identification function (10a) to measure the physiological signal and identify a user identity concurrently, without using the external electronic device 90. The physiological signal measuring apparatus with identification function (10a) comprises the measuring module 11, the identifying module 12, a receiving module 15, a comparing module 16, and a storing module 17. The measuring module 11 obtains the physiological signal of the user 80 and produces the physiological datum 50 according to the physiological signal. The identifying module 12 obtains the identification datum 60 of the user 80. The receiving module 15 receives the physiological datum 50 and the identification datum 60. The comparing module 16 searches the identity identification database 19 to identify a user identity according to the identification datum 60. The storing module 17 stores the physiological datum 50 in the corresponding physiological data region in the physiological database 18 according to the user identity datum.

A point to note is that, in an embodiment of the present invention, the aforesaid elements can not only come in the form of a hardware device, a software program, firmware, or a combination thereof, but also come in the form of a circuit loop or any other appropriate arrangement. If any one of the elements is implemented by software, it can be loaded on a computer-readable storage medium. Each of the elements can be self-contained; alternatively, the elements can operate in conjunction with each other. Furthermore, this embodiment is intended to be illustrative of a preferred embodiment of the present invention, and is not described in detail in terms of any possible combination of variations for the sake of brevity. However, persons skilled in the art should be able to understand that the present invention can, for the purpose of the implementation thereof, require any other more delicate conventional modules or elements. The modules or elements can be omitted or altered as needed. Any other module or element is not necessarily absent between any two of the modules.

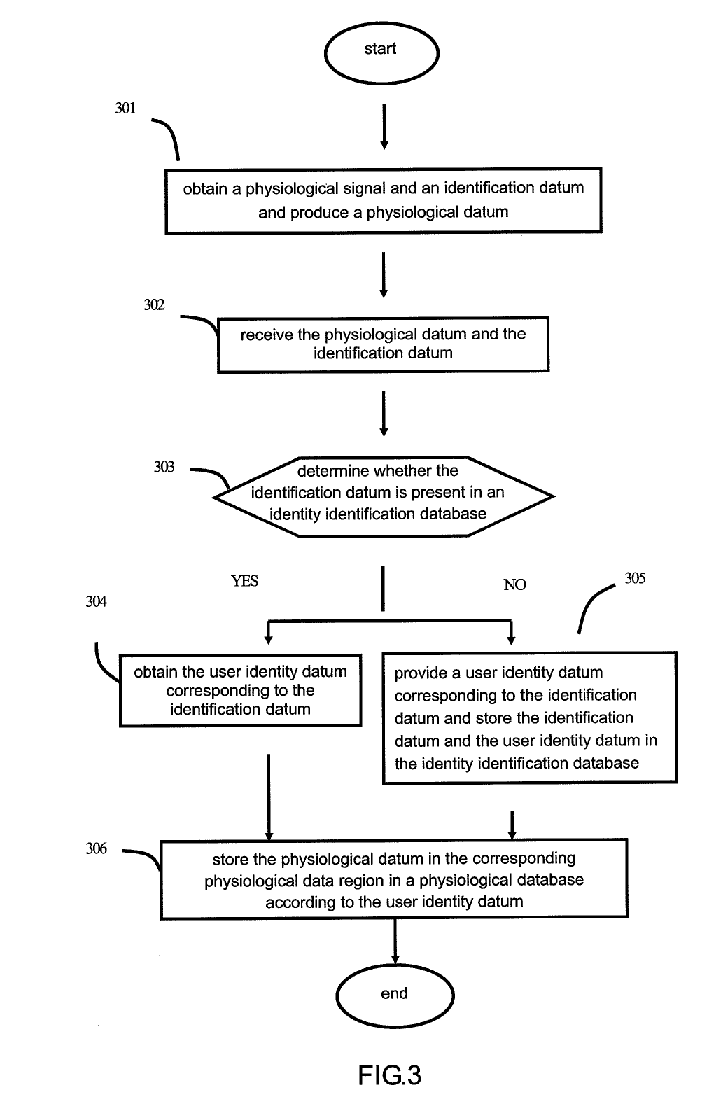

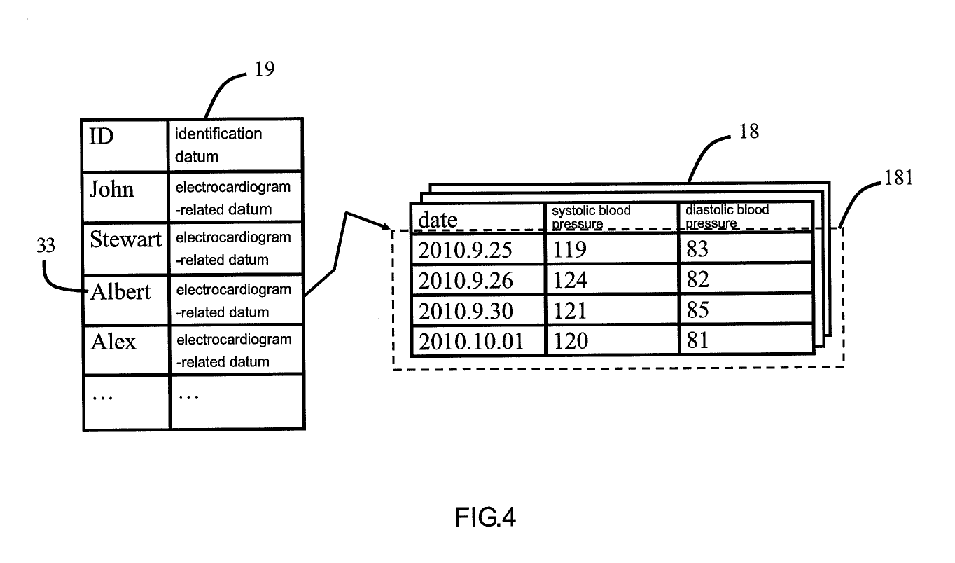

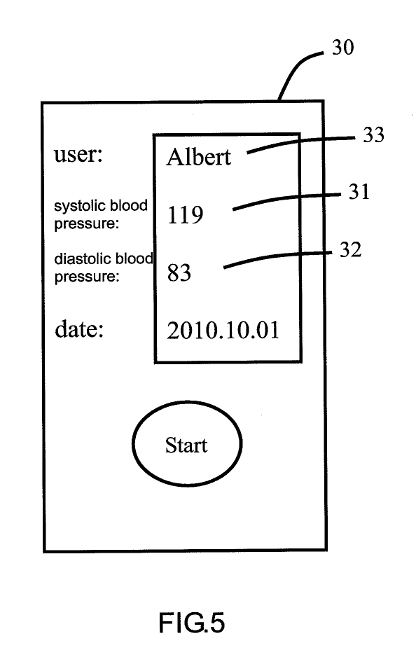

Referring to FIG. 3, FIG. 4, and FIG. 5, there is shown in FIG. 3 a flowchart of a physiological signal measuring method with identification function according to a specific embodiment of the present invention. Although the physiological signal measuring method with identification function of the present invention is illustrated hereunder with the physiological signal measuring apparatus with identification function (10a) shown in FIG. 2, the physiological signal measuring method with identification function of the present invention is not limited to its application in the physiological signal measuring apparatus with identification function (10a).

Step 301: obtain a physiological signal and an identification datum and produce a physiological datum.

Step 301 is executed by the measuring module 11 and the identifying module 12. The user 80 measures the physiological signal with the measuring module 11. The measuring module 11 obtains the physiological signal and then produces the physiological datum 50. The identification datum 60 is obtained by means of the identifying module 12. In this embodiment, the user 80 measures blood pressure with the measuring module 11 and then produces a blood pressure level, such that the blood pressure level thus produced functions as the physiological datum 50. A feature parameter of an electrocardiogram of the user 80 is obtained by means of the identifying module 12, such that the obtained feature parameter functions as the identification datum 60. A point to note is that the physiological signal is not limited to a blood pressure-related signal, because the physiological signal can also be a blood sugar-related signal, an electrocardiogram-related signal, or any other physiological signal. Likewise, the physiological datum 50 is not limited to a blood pressure level, because the physiological datum 50 can also be a blood sugar level, an electrocardiogram-related datum, or any other physiological datum. Likewise, the identification datum 60 is not limited to an electrocardiogram-related datum, because the identification datum 60 can also be a fingerprint-related datum, an iris-related datum, a facial feature-related datum, or any other identifiable datum. Also, a way of obtaining a physiological signal and producing a physiological datum and a way of obtaining the identification datum 60 are attributable to the prior art but are not regarded as a focus of the present invention, and thus they are not described in detail herein for the sake of brevity.

Step 302: receive the physiological datum and the identification datum.

The receiving module 15 receives the identification datum 60 and the physiological datum 50 of the user 80. In this embodiment, the receiving module 15 receives a systolic blood pressure 31 and a diastolic blood pressure 32 of the user 80 and receives an electrocardiogram-related datum of the user 80. The systolic blood pressure 31 and the diastolic blood pressure 32 thus received function as the physiological datum 50. The electrocardiogram-related datum thus received functions as the identification datum 60.

Step 303: determine whether the identification datum is present in an identity identification database.

Upon receipt of the physiological datum 50 and the identification datum 60, the comparing module 16 determines whether the identification datum 60 is present in the identity identification database 19. Referring to FIG. 4, there is shown a schematic view of the physiological database and the identity identification database 19 according to the present invention. User IDs and the identification datum 60 (that is, the electrocardiogram-related datum in this embodiment) corresponding to the user IDs, respectively, are stored in the identity identification database 19; hence, the comparing module 16 can determine whether the electrocardiogram-related datum (that is, the identification datum 60) of the user 80 is present in the identity identification database 19. A way of determining whether the identification datum is present in the identity identification database 19 is attributable to the prior art but is not regarded as a focus of the present invention, and thus it is not described in detail herein for the sake of brevity.

If the identification datum is present in the identity identification database, then go to step 304: obtain the user identity datum corresponding to the identification datum.

If the identification datum 60 is present in the identity identification database 19, the comparing module 16 can directly obtain the user identity datum corresponding to the identification datum 60. In this embodiment, assuming that the comparing module 16 discovers that the electrocardiogram-related datum (that is, the identification datum 60) of the user 80 is present in the identity identification database 19, the user identity datum 33 (Albert) corresponding to the identification datum 60 can be obtained.

If the identification datum is not present in the identity identification database, then go to step 305: provide a user identity datum corresponding to the identification datum and store the identification datum and the user identity datum in the identity identification database.

Assuming, in step 303, that the comparing module 16 discovers that the identification datum 60 is not present in the identity identification database 19, the comparing module 16 will provide the user identity datum (“Albert”, for example) corresponding to the identification datum 60 and store the identification datum 60 and the user identity datum corresponding thereto in the identity identification database 19.

Step 306: store the physiological datum in the corresponding physiological data region in a physiological database according to the user identity datum.

After the user identity has been identified, the physiological datum 50 of the user 80 can be stored in a data block of the physiological database by means of the storing module 17, wherein the data block corresponds to the user 80. Referring to FIG. 4 and FIG. 5, in this embodiment, the storing module 17 stores the systolic blood pressure 31 and the diastolic blood pressure 32 of the user 80 in a corresponding physiological data region 181 in the physiological database 18 for use in subsequent comparison, wherein the corresponding physiological data region 181 is attributed to the user identity datum 33. The systolic blood pressure 31, the diastolic blood pressure 32, and the user identity datum 33 are displayed on an operating interface 30. Ways of identifying a user identity according to an electrocardiogram-related datum are disclosed in academic papers published by local academics and overseas academics, and the identification datum 60 is not limited to an electrocardiogram-related datum; hence, possible ways are not described in detail herein for the sake of brevity.

The aforesaid embodiments illustrate how the user directly uses the physiological signal measuring apparatus with identification function (10a) to measure a physiological signal and identify a user identity concurrently. Nonetheless, the physiological signal measuring apparatus with identification function of the present invention can also identify a user identity through the external electronic device 90. The physiological signal measuring method with identification function, which is performed with the physiological signal measuring apparatus with identification function (10) shown in FIG. 1, effectuates identification of a user identity through the external electronic device 90, by following the steps as follows:

Step 301: obtain a physiological signal and an identification datum and produce a physiological datum.

Step 301 is executed by the measuring module 11 and the identifying module 12. In this embodiment, the user 80 measures an electrocardiogram through the measuring module 11, produces an electrocardiogram-related datum that functions as the physiological datum 50, and obtains a fingerprint-related datum of the user 80 through the identifying module 12, wherein the obtained fingerprint-related datum functions as the identification datum 60.

Step 302: receive the physiological datum and the identification datum.

After obtaining the physiological signal and the identification datum 60 and producing the physiological datum 50, it is feasible for the transmitting module 13 to transmit the aforesaid datum to the external electronic device 90, such that the external electronic device 90 receives the aforesaid datum and processes them. In this embodiment, the transmitting module 13 transmits an electrocardiogram-related datum and a fingerprint-related datum to the external electronic device 90.

Step 303: determine whether the identification datum is present in an identity identification database.

After the external electronic device 90 has received the aforesaid datum, the processor 91 executes the identifying program 100 in the memory 92 to determine whether the identification datum 60 is present in the identity identification database 19. In this embodiment, the identifying program 100 determines whether the fingerprint-related datum is present in the identity identification database 19.

If the identification datum 60 is present in the identity identification database, then go to step 304: obtain the user identity datum corresponding to the identification datum.

If the identification datum 60 is present in the identity identification database 19, the external electronic device 90 can directly obtain the user identity datum corresponding to the identification datum 60. In this embodiment, assuming that the external electronic device 90 discovers that the fingerprint-related datum of the user 80 is present in the identity identification database 19, the user identity datum corresponding to the identification datum 60 can be obtained.

If the identification datum 60 is not present in the identity identification database 19, then go to step 305: provide a user identity datum corresponding to the identification datum and store the identification datum and the user identity datum in the identity identification database.

Assuming, in step 303, that the external electronic device 90 discovers that the identification datum 60 is not present in the identity identification database 19, the external electronic device 90 will provide a user identity datum corresponding to the identification datum 60 (that is, the fingerprint-related datum of the user 80) and store the identification datum 60 and the user identity datum corresponding thereto in the identity identification database 19.

Step 306: store the physiological datum in the corresponding physiological data region in a physiological database according to the user identity datum.

After the user identity has been identified, the processor 91 executes a recording program 101 in the memory 92 and stores the physiological datum 50 of the user in a data block of the physiological database, wherein the data block corresponds to the user. In this embodiment, an electrocardiogram-related datum of the user 80 is stored in a data block of the physiological database 18 for use in subsequent comparison, wherein the data block corresponds to the user 80.

A point to note is that the physiological signal measuring method with identification function of the present invention is not limited to the aforesaid sequence of the steps; instead, the aforesaid sequence of the steps can be subject to changes, provided that the objectives of the present invention are achieved.

In conclusion, the features of the present invention are completely different from those of the prior art in terms of objectives, means, and effects. However, it should be noted that the above embodiments are illustrative of the principle and effect of the present invention only, and should not be interpreted as restrictive of the scope of the present invention. Hence, persons skilled in the art can make modifications and changes to the aforesaid embodiments without going against the technical principle and spirit of the present invention. Accordingly, the extent of legal protection for the rights claimable toward the present invention should be defined by the appended claims.

What is claimed is:

1. A physiological signal measuring apparatus with identification function, the apparatus being electrically connected to a computer, the apparatus comprising:
a measuring module for obtaining a physiological signal of a user and producing a physiological datum according to the physiological signal;
an identifying module for obtaining an identification datum of the user; and
a transmitting module electrically connected to the measuring module and the identifying module for transmitting the physiological datum and the identification datum to the computer.

2. The apparatus of claim 1, wherein the identification datum is one of a set of an electrocardiogram-related datum, a fingerprint-related datum, a facial feature-related datum, and an iris-related datum.

3. The apparatus of claim 2, wherein the physiological signal is one of a set of a blood pressure-related signal, a blood sugar-related signal, and an electrocardiogram-related signal, and the physiological datum is one of a set of a blood pressure level, a blood sugar level, and the electrocardiogram-related datum.

4. The apparatus of claim 1, wherein the physiological signal is one of a set of a blood pressure-related signal, a blood sugar-related signal, and an electrocardiogram-related signal, and the physiological datum is one of a set of a blood pressure level, a blood sugar level, and an electrocardiogram-related datum.

5. A physiological signal measuring method with identification function, the method comprising the steps of:
receiving an identification datum and a physiological datum of a user;
determining whether the identification datum is present in an identity identification database, so as to perform one of the two steps of:
providing a user identity datum corresponding to the identification datum and storing the identification datum and the user identity datum in the identity identification database if it is determined that the identification datum is not present in the identity identification database; or
obtaining the user identity datum corresponding to the identification datum if it is determined that the identification datum is present in the identity identification database;
and
storing the physiological datum in a corresponding physiological data region in a physiological database according to the user identity datum.

6. The method of claim 5, wherein the identification datum is one of a set of an electrocardiogram-related datum, a fingerprint-related datum, a facial feature-related datum, and an iris-related datum.

7. The method of claim 6, wherein the physiological datum is one of a set of a blood pressure level, a blood sugar level, and the electrocardiogram-related datum.

8. The method of claim 5, wherein the physiological datum is one of a set of a blood pressure level, a blood sugar level, and an electrocardiogram-related datum.

9. A physiological signal measuring apparatus with identification function, the apparatus comprising:
a receiving module for receiving an identification datum and a physiological datum of a user;
a comparing module electrically connected to the receiving module for determining whether the identification datum is present in an identity identification database, so as to perform one of the two mechanisms of:
providing a user identity datum corresponding to the identification datum and storing the identification datum and the user identity datum in the identity identification database if it is determined that the identification datum is not present in the identity identification database; or
obtaining the user identity datum corresponding to the identification datum if it is determined that the identification datum is present in the identity identification database;
and
a storing module electrically connected to the comparing module for storing the physiological datum in a corresponding physiological data region in a physiological database according to the user identity datum.

10. The apparatus of claim 9, further comprising:
a measuring module for obtaining a physiological signal of the user and producing the physiological datum according to the physiological signal;
and
an identifying module for obtaining the identification datum of the user.

11. The apparatus of claim 10, wherein the identification datum is one of a set of an electrocardiogram-related datum, a fingerprint-related datum, a facial feature-related datum, and an iris-related datum.

12. The apparatus of claim 11, wherein the physiological signal is one of a set of a blood pressure-related signal, a blood sugar-related signal, and an electrocardiogram-related signal, and the physiological datum is one of a set of a blood pressure level, a blood sugar level, and the electrocardiogram-related datum.

13. The apparatus of claim 10, wherein the physiological signal is one of a set of a blood pressure-related signal, a blood sugar-related signal, and an electrocardiogram-related signal, and the physiological datum is one of a set of a blood pressure level, a blood sugar level, and an electrocardiogram-related datum.
